(19) United States
(12) Patent Application Publication
(10) Pub. No.: US 2004/0022834 A1
(43) Pub. Date: Feb. 5, 2004

(54) Pharmaceutical oxan preparation

(76) Inventors: Joel Bougaret, Francarville (FR); Laurent Liorzou, L'Union (FR); Michel Sournac, Toulouse (FR)

Correspondence Address: THE FIRM OF HUESCHEN AND SAGE, 500 COLUMBIA PLAZA, 350 EAST MICHIGAN AVENUE, KALAMAZOO, MI, 49007, US

(21) Appl. No.: 10/381,801

(22) PCT Filed: Sep. 26, 2001
(86) PCT No.: PCT/FR01/02980

(30) Foreign Application Priority Data
Sep. 26, 2000 (FR) FR0012197

(51) Int. Cl. A61L 9/04; A61K 9/70

(52) U.S. Cl. 424/449; 424/45

(57) ABSTRACT

The invention concerns a topical pharmaceutical preparation, characterized in that it consists of the combination of an oxan and a pharmaceutically acceptable excipient for transdermal delivery of said oxan.

The present invention relates, in a general way, to topical pharmaceutical preparations consisting of the combination of an active ingredient, namely an oxan, and a pharmaceutically acceptable excipient allowing transdermal administration of said active ingredient.

In the context of the present application, the expression oxan will denote any imidazoline derivatives endowed with antagonist properties for the α₂-adrenergic receptors and in particular:

idazoxan or 2-(1,4-benzodiosan-2-yl)-2-imidazoline in the form of the free base or the hydrochloride (as described in Patent GB 2 068 376);

alkoxyidazoxans, that is to say 2-(2-alkoxy-1,4-benzodioxan-2-yl)-2-imidazolines), in particular 2-methoxy- and 2-ethoxyidazoxan (as described in Patent EP 92 328);

the fluorinated derivatives of benzodioxane-imidazolines of formula:

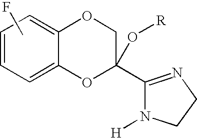

in which:

R represents a linear, branched or cyclized alkyl or alkenyl group comprising 1 to 7 carbon atoms, or a benzyl group, and

the fluorine atom can occupy the 5-, 6-, 7- or 8-position (as described in French Patent Application No. 99 08302);

the 2,3-dihydroxybenzofuran derivatives which are disubstituted at the 2-position such as efaroxan, that is to say 2-[2-(2-ethyl-2,3-dihydroxybenzofuranyl)]-2-imidazoline, or dexefaroxan which may be prepared by the method described in International Patent Application WO-0002836.

The various oxans which may enter into the composition of the pharmaceutical preparations which are the subject of the present invention may be used in the form of their racemic mixtures, of various forms or enantiomeric mixtures, in the form of the free base or alternatively the acid addition salts with pharmacologically acceptable acids, in particular the hydrochloride.

The present invention therefore relates to such topical pharmaceutical preparations which may be provided in various galenic forms, in particular in the form of creams, ointments, gels, film-forming aerosol or spray dispensers, or alternatively in the form of a transdermal matrix patch.

More particularly, the subject of the present invention is a transdermal, pharmaceutical preparation containing, as active ingredient, efaroxan or 2-[2-(2-ethyl-2,3-dihydrobenzofuranyl)]-2-imidazoline in the form of the base or the hydrochloride, and the method for producing this preparation. More specifically, the present invention relates to a transdermal patch formulated so as to allow the absorption of efaroxan through the skin, the latter making it possible to prolong the therapeutic activity of this molecule. This matrix device comprises a support, a polymeric adhesive matrix and a protectant.

The transdermal route is a route of administration which is accepted in order to overcome the drawbacks of other routes of administration, and in particular in order to allow the progressive installation of uniform plasma levels, in the case of repeated administration. Furthermore, this route of administration improves patient compliance with the treatment, which is more particularly true in the case of patients who may require an efaroxan-based therapy.

Efaroxan is a potent and selective antagonist compound for α₂-adrenergic receptors, whose therapeutic uses could be of benefit either in the treatment of Alzheimer's disease, or of that of progressive supranuclear paralysis (PSP).

For any medicament, in general, a sharp rise in plasma levels is capable of causing the onset of undesirable effects; in the case of efaroxan, they are of the cardiovascular type.

Finally, by virtue of their nature, the molecules are absorbed to a greater or lesser degree through the human skin; in the case of dexefaroxan, preliminary studies of skin permeation in vitro on animal skins have demonstrated the existence of significant flows, making it possible to envisage, as probable, the possibility of administering therapeutic quantities in humans by this route. Indeed, using aqueous-alcoholic solutions (10% m/v), quantities accumulated at 24 hours were demonstrated, respectively of the order of 8.5 mg/cm² for the base form, and of the order of 1.0 mg/cm² for the hydrochloride form. These values are sufficiently high to make it possible to aim at the daily administration, from a transdermal patch, of a quantity of efaroxan base or hydrochloride in the mg range. Mention may also be made of the case of idazoxan, for which, using aqueous-alcoholic solutions (10% m/v), quantities accumulated at 24 hours were demonstrated, respectively of the order of 5 mg/cm² for the base form and of the order of 0.25 mg/cm² for the hydrochloride form.

Thus, with the aim of improving the possibilities of therapeutic treatment based on oxans, the present invention relates in particular to a novel transdermal galenic form and its method of preparation.

According to the invention, there is therefore proposed the production of adhesive matrix devices for the administration of oxans, and in particular of dexefaroxan in the base or hydrochloride form. This invention groups together, on the one hand, several formulations, and, on the other hand, their method of manufacture.

The previously stated objective is obtained by virtue of a novel matrix-type formulation containing the active ingredient, an adhesive matrix and formulation excipients. More precisely, there is recommended, according to the invention, an adhesive device for the systemic administration of dexefaroxan or of one of its salts, said system comprising a support, a protectant and an active self-adhesive matrix (ASAM), characterized by:

a) 40 to 95 parts by weight of copolymers consisting of ASAM;

b) 5 to 40 parts by weight of processing plasticizers or solvents;

c) 5 to 20 parts by weight of an oxan;

d) 0 to 20 parts by weight of solubilizing agent and/or permeation adjuvant.

According to a particular characteristic of the invention, the adhesive copolymer is composed either of one or more adhesive polyacrylates, or of one or more hydrophilic polymers, or of silicone polymers.

This adhesive copolymer may be an adhesive acrylic copolymer consisting of at least two of the monomers chosen from the products designated hereinafter, acrylic acid, butyl acrylate, 2-ethylhexyl acrylate, vinyl acetate, methyl acrylate, glycidyl methacrylate, 2-hydroxyethyl acrylate, methyl methacrylate, n-vinylpyrrolidone, butyl methacrylate, methacrylic esters and dimethylaminoethyl methacrylate.

According to a particular characteristic of the invention, the hydrophilic polymer is obtained by combining polyvinyl alcohol and polyvinylpyrrolidone.

According to a particular characteristic of the invention, the plasticizing adjuvant forms part either of the family of mineral oils such as glycerol, or of products obtained by polymerization of ethylene, and preferably of the polyethylene glycol type having a molecular mass of between 200 and 8 000.

According to a particular characteristic of the invention, polydimethylsiloxane, obtained by condensation of a silanol with a silicate resin, is used as polymer.

According to a particular embodiment of the matrix device according to the invention, the matrix comprises, for a total of 100 parts by weight:

a) 60 to 80 parts by weight of an adhesive and self-crosslinkable acrylic copolymer, in the form of a solution containing about 47.5% w/v of 2-ethylhexyl acrylate, glycidyl methacrylate, 2-hydroxyethyl acrylate and vinyl acetate copolymer and, as crosslinking agent, polybutyl titanate, said “ready-to-use” adhesive copolymer having a glass transition temperature of −50° C.;

b) 0.5 to 10 parts by weight of polyvidone;

c) 5 to 15 parts by weight of dexefaroxan in hydrochloride form;

d) 0 to 2 parts by weight of antioxidant;

e) 0 to 15 parts by weight of one or more absorption promoters.

According to another particular embodiment of the matrix device according to the invention, the matrix comprises, for a total of 100 parts by weight:

a) 60 to 80 parts by weight of an adhesive and self-crosslinkable acrylic copolymer, in the form of a solution containing about 47.5% w/v of acrylic acid, butyl acrylate, 2-ethylhexyl acrylate and vinyl acetate copolymer and, as crosslinking agent, aluminum acetylacetonate, said “ready-to-use” adhesive copolymer having a glass transition temperature of −50° C.;

b) 0.5 to 10 parts by weight of polyvidone;

c) 5 to 15 parts by weight of dexefaroxan in hydrochloride form;

d) 0 to 2 parts by weight of antioxidant;

e) 0 to 15 parts by weight of one or more absorption promoters.

According to another particular embodiment of the matrix device according to the invention, the matrix comprises, for a total of 100 parts by weight:

a) 30 to 60 parts by weight of an adhesive acrylic copolymer, in the form of a solution containing about 60% w/v of dimethylaminoethyl methacrylate and methacrylic ester copolymer, and, as crosslinking agent, succinic acid, and, as plasticizer, acetyltributyl citrate;

b) 0.5 to 10 parts by weight of polyvidone;

c) 5 to 10 parts by weight of dexefaroxan in hydrochloride form;

d) 0 to 2 parts by weight of antioxidant;

e) 0 to 15 parts by weight of one or more absorption promoters.

According to another particular embodiment of the matrix device according to the invention, the matrix comprises, for a total of 100 parts by weight:

a) 5 to 20 parts by weight of polyvinyl alcohol;

b) 20 to 60 parts by weight of polyvidone;

c) 10 to 30 parts by weight of glycerol;

d) 10 to 30 parts by weight of polyethylene glycol;

e) 5 to 15 parts by weight of dexefaroxan in hydrochloride form;

f) 0 to 2 parts by weight of antioxidant;

g) 0 to 15 parts by weight of one or more absorption promoters.

According to another particular embodiment of the matrix device according to the invention, the matrix comprises, for a total of 100 parts by weight:

a) 70 to 95 parts by weight of silicone polymer;

b) 5 to 15 parts by weight of dexefaroxan in hydrochloride form;

c) 0 to 2 parts by weight of antioxidant;

d) 0 to 15 parts by weight of one or more absorption promoters.

According to another particular embodiment of the matrix device according to the invention, the topical formulation comprises, for a total of 100 parts by weight:

a) 5 to 30 parts by weight of polysaccharide polymer;

b) 1 to 15 parts by weight of dexefaroxan in hydrochloride form;

c) 5 to 30 parts by weight of ethanol;

d) 20 to 60 parts by weight of water;

e) 0 to 15 parts by weight of one or more absorption promoters.

According to another particular embodiment of the matrix device according to the invention, the topical formulation comprises, for a total of 100 parts by weight:

a) 2 to 40 parts by weight of carbomer;

b) 1 to 15 parts by weight of dexefaroxan in hydrochloride form;

c) 5 to 25 parts by weight of isopropyl alcohol;

d) 5 to 25 parts by weight of polyoxyethylene alkyl ether;

e) 5 to 25 parts by weight of fatty acid esters;

f) 5 to 25 parts by weight of fatty alcohol esters;

g) 0 to 15 parts by weight of one or more absorption promoters.

According to an additional characteristic of the present invention, the matrix device comprises, in addition, one or more absorption promoters, preferably selected from alcohols, glycols, polyglycols, amides of the pyrrolidone type and derivatives, surfactants of the nonionic type, polysorbates, alkyl ethers, aryl ethers, poloxamers, saturated or unsaturated fatty acids with a carbon chain between C₅ and C₃₀, fatty alcohols, polyglycosylated glycerides, alone or as mixtures, glycol esters of propylene glycol or of polyglycerol, fatty acid esters of the polyol type, alkylglyceryl ether, propylene glycol, glycerine, polyoxyethylene glycerol, polyglycerol, sorbitan, polyoxyethylene sorbitan, polyoxyethylene castor oil, alkyl ether, esters of sugars, derivatives of collagens, terpenic essential oils, compounds of the m-diethyltoluamide type, antipuriginous compounds of the crotamiton type, compounds of the phospholipid type, lecithin derivatives, neohesperidin dihydro-chalcone derivatives.

The present invention also relates to a method for preparing a transdermal adhesive matrix patch involving the use of the following successive steps:

preparing a premixture of active ingredient in the cosolvent(s) for the adhesive or in an additional processing solvent, in order to obtain either a solution, or a dispersion;

adding to the preceding premixture the required quantities of plasticizers and adhesive;

placing the mixture directly on a supporting film, preferably of the silicone polyester type, so as to obtain a layer having a thickness of between 50 and 100 g/m² (expressed as dry weight);

drying the coating thus obtained in order to evaporate the processing solvents and to allow crosslinking of the polymers, by progressive drying at a temperature of between 50° C. and 110° C., and preferably via different drying methods;

pasting onto the dried coating an occlusive film, for example of the polyester type;

cutting out to the desired surface and packaging in a sachet.

Finally, the present invention also extends, in a general way, to the use of an oxan as defined above for the manufacture of a topical pharmaceutical preparation intended for the treatment of lipolysis and of obesity or alternatively for the treatment of Alzheimer's disease, of progressive supranuclear paralysis (PSP), of Parkinson's disease and/or of depression.

There will preferably be recourse to a topical pharmaceutical preparation in the form of a cream, ointment, gel, film-forming aerosol or spray dispenser for the treatment of lipolysis and of obesity, while for the treatment of Alzheimer's disease, progressive supranuclear paralysis, Parkinson's disease and depression, a transdermal matrix patch will be more readily used.

According to the invention, a method of manufacture for the preparation of said transdermal matrix patch is also recommended, which involves the succession of the following steps which are described in greater detail:

i) preparing a premixture of active ingredient in the cosolvent(s) for the adhesive or in an additional processing solvent;

ii) adding to the premix i) the required quantities of plasticizers and of adhesive or nonadhesive polymers;

iii) depositing the mixture ii) directly on a supporting film, preferably of the silicone polyester type, so as to obtain a layer having a thickness of between 50 and 100 g/m² (expressed as dry weight);

iv) drying the coating thus obtained in order to evaporate the processing solvents and to allow crosslinking of the polymers, by progressive drying at a temperature of between 50° C. and 110° C., and preferably via different drying methods; in the coating, the active ingredient thus exists either in the form of a solution, or in the form of a molecular dispersion;

v) pasting onto the dried coating an occlusive film, for example of the polyester type.

Preferably, the following compounds will be used, alone or in combination, to constitute ASAM:

a) either an acrylic copolymer having a low average molecular mass, with an alcohol functionality, characterized by the presence of 4 base monomers such as 2-ethylhexyl acrylate, glycidyl methacrylate, 2-hydroxyethyl acrylate and vinyl acetate. This acrylic copolymer (for example DURO-TAK® 387-2516 from the company National Starch & Chemical) is a “ready-to-use” self-crosslinked adhesive available in the form of an organic solution having a theoretical density close to 0.90 g/cm³, a relative viscosity of between 3.1 and 4.2, and a mean viscosity on Brookfield (at 25° C., 12 rpm, rotor No. 3) between 2 700 and 6 000 mPa.s;

b) or an acrylic copolymer having a low average molecular mass, with a carboxylic functionality, characterized by the presence of 4 base monomers such as acrylic acid, butyl acrylate, 2-ethylhexyl acrylate and vinyl acetate. This acrylic copolymer (for example DURO-TAK® 387-2052 from the company National Starch & Chemical) is a “ready-to-use” self-crosslinked adhesive available in the form of an organic solution having a theoretical density close to 0.92 g/cm³, a relative viscosity of between 2.5 and 2.9, and a mean viscosity on Brookfield (at 25° C., 12 rpm, rotor No. 3) between 1 500 and 4 000 mPa.s;

c) or a cationic acrylic copolymer, characterized by the presence of monomers of the dimethylaminoethyl methacrylate and methacrylic ester type. This acrylic copolymer (for example EUDRAGIT E100® from the company Rohm) is available in the form of a granule whose use in organic medium also requires the addition of plasticizers and of crosslinking agents. The product thus obtained has good adhesion properties, at a mean viscosity on Brookfield (at 20° C., II/6) between 800 and 1 000 mPa.s. Its dry matter content is close to 60%;

d) or a hydrophilic polyvinylpyrrolidone polymer and more particularly of the 1-vinyl-2-pyrrolidone type having a molecular mass of between 30 000 and 1 500 000, or a combination of these same compounds. In the soluble form, these products (for example KOLLIDON 30®, KOLLIDON 12F® and KOLLIDON 90F® from the company BASF) are available in the form of hygroscopic powders of different particle size and allow the manufacture of a “dry” matrix whose adhesivity will develop after preliminary moistening of the skin and will develop during the application period. When they are not used for their water-solubility properties, these polymers can increase the stability of ASAM by acting on the free volume of acrylic copolymers;

e) or a hydrophilic polyvinyl alcohol polymer having a molecular mass between 30 000 and 200 000 or a combination of these same compounds. In the soluble form, these products (for example RHODOVIOL 25/140® from the company Rhône-Poulenc) are available in the form of hygroscopic powders which are used for their properties as viscosifying agents, nonionic surfactants or film-forming agents. In the present case, they participate in the manufacture of a “dry” matrix whose adhesivity will develop after preliminary moistening of the skin and will develop during the application period;

f) or a polymer belonging to the silicone group. In a soluble form, these products (for example BIO-PSA®X7-4602 from the company Dow Corning) are available in the form of a solution in ethyl acetate, having a mean viscosity of between 800 and 1 200 mPa.s;

g) or a polymer belonging to the partially substituted cellulose ether family, such as hydroxyethylcellulose, hydroxypropylcellulose, carboxymethylcellulose sodium, or to that of polysaccharide gums. In a soluble form, these products (for example RHODIGEL 200 from the company Rhodia) are available in molecular masses of between 80 000 and 1 200 000, which allow aqueous-alcoholic gels of varying viscosity to be obtained;

h) or a polymer belonging to the carbomer group; these synthetic polymers are obtained by copolymerization of acrylic acid with allylsucrose or allyl ethers of pentaerythritol. After having been neutralized, these carbomers (for example CARBOMER 943P from the company BF Goodrich) allow aqueous-alcoholic gels of varying viscosity and texture to be obtained.

Among the formulation adjuvants which are suitable according to the invention, there may be mentioned the use of hydrophilic plasticizers capable of stabilizing the dispersion of the active ingredient in the polymers, or of improving the skin tolerance of the transdermal patch. There may be preferably mentioned polyethylene glycols and glycerol, alone or in combination.

The support which protects the adhesive matrix may be any support generally used in the formulation of transdermal patches, which is occlusive and inert toward the constituents of the matrix. Among the products generally used, there may be mentioned polyethylene, polypropylene and polyester films, multilayer complexes consisting of the above materials combined, for example, with fine aluminum layers, combinations of vinyl acetate and ethylene copolymers in the form of films or of a foam. Preferably, there will be used either a polyester support film, or a multilayer complex of the low-density polyethylene type and polyvinylidene chloride type.

The protectant which constitutes the disposable part of the adhesive device before application is generally a product having good cutting properties, and which is inert toward the components of the matrix; there should be mentioned, among the products that are used most often, paper, polyester and polyvinyl chloride films. Preferably, a siliconized or fluorinated polyester film which will be cut beforehand in order to facilitate its removal before application will be used.

The final device will be packaged using an impervious protection of the sachet type with the aid of polyethylene-aluminum complex films, or of the blister type.

According to the invention, a variation is also recommended which consists in the presence of an antioxidant or of sequestering agents which, in the form of adjusted quantities, enhance the stability of the composition.

According to the invention, a variation is also recommended which consists in the presence of one or more absorption promoters and/or solubilizing agents which, in the form of adjusted quantities, facilitate skin permeation by the active ingredient or its solubilization in the adhesive matrix.

The examples below illustrate the invention without, however, limiting it:

### EXAMPLE 1

By mixing dexefaroxan hydrochloride in the adhesive, adding other ingredients, coating, drying and cutting, a composition is prepared which comprises:

| | |
|---|---|
| Dexefaxoran hydrochloride | 15.4 mg (equivalent to 10% |
| | m/m base) |
| Duro-Tak 387-2516 ® | 131.3 mg |
| Kollidon 12PF ® | 7.7 mg |
| Support Hostaphan RN 23 MED | 20 cm² |
| Protectant Gelroflex 75 μm | 20 cm² |

### EXAMPLE 2

By mixing dexefaroxan hydrochloride in the adhesive, adding other ingredients, coating, drying and cutting, a composition is prepared which comprises:

| | |
|---|---|
| Dexefaxoran hydrochloride | 16.5 mg (equivalent to 10% |
| | m/m base) |
| Duro-Tak 387-2052 ® | 138.9 mg |
| Kollidon 12PF ® | 8.1 mg |
| Support Hostaphan RN 23 MED | 20 cm² |
| Protectant Gelroflex 75 μm | 20 cm² |

### EXAMPLE 3

By mixing dexefaroxan hydrochloride in the adhesive, adding other ingredients, coating, drying and cutting, a composition is prepared which comprises:

| | |
|---|---|
| Dexefaxoran hydrochloride | 16.0 mg (equivalent to 10% |
| | m/m base) |
| Eudragit E100 | 87.4 mg |
| Acetyltributyl citrate | 43.9 mg |
| Kollidon 12PF ® | 8.0 mg |
| Succinic acid | 3.4 mg |
| Support Hostaphan RN 23 MED | 20 cm² |
| Protectant Gelroflex 75 μm | 20 cm² |

### EXAMPLE 4

By mixing dexefaroxan hydrochloride in the adhesive, adding other ingredients, coating, drying and cutting, a composition is prepared which comprises:

| | |
|---|---|
| Dexefaxoran hydrochloride | 16.6 mg (equivalent to 10% |
| | m/m base) |
| Glycerol | 29.6 mg |
| Macrogol 600 P | 30.2 mg |
| Polyvidone K30 | 44.6 mg |
| Polyvidone K90 | 22.3 mg |
| Rhodoviol 25/140 | 21.2 mg |
| Support Hostaphan RN 23 MED | 20 cm² |
| Protectant Gelroflex 75 μm | 20 cm² |

### EXAMPLE 5

By mixing dexefaroxan hydrochloride in the adhesive, adding other ingredients, coating, drying and cutting, a composition is prepared which comprises:

| | |
|---|---|
| Dexefaxoran hydrochloride | 15.7 mg (equivalent to 10% |
| | m/m base) |
| BioPsa MD7-4602 | 140.9 mg |
| Support Hostaphan RN 23 MED | 20 cm² |
| Protectant Gelroflex 75 μm | 20 cm² |

### EXAMPLE 6

By mixing dexefaroxan hydrochloride in the other ingredients, homogenizing and packaging, a composition is prepared which comprises:

| | |
|---|---|
| Dexefaroxan hydrochloride | 1 g (equivalent to 1% m/m |
| | base) |
| Rhodigel 200 | 7 g |
| Nipagin | 0.25 g |
| Nipasol | 0.25 g |
| Ethyl alcohol at 95% v/v | 42.0 g |
| Water qs | 100 g |

### EXAMPLE 7

By mixing dexefaroxan hydrochloride in the other ingredients, homogenizing and packaging, a composition is prepared which comprises:

| | |
|---|---|
| Dexefaxoran hydrochloride | 1 g (equivalent to 1% m/m |
| | base) |
| Carbomer P 934 | 15 g |
| Triethanolamine | 0.5 g |
| Isopropyl alcohol | 43.5 g |
| Liquid paraffin | 10 g |
| Cetiol | 15 g |
| Cetomacrogol 1000 | 15 g |

Other aspects of the invention will emerge on reading the detailed description which follows. The invention will also be understood more clearly with the aid of the accompanying drawings in which:

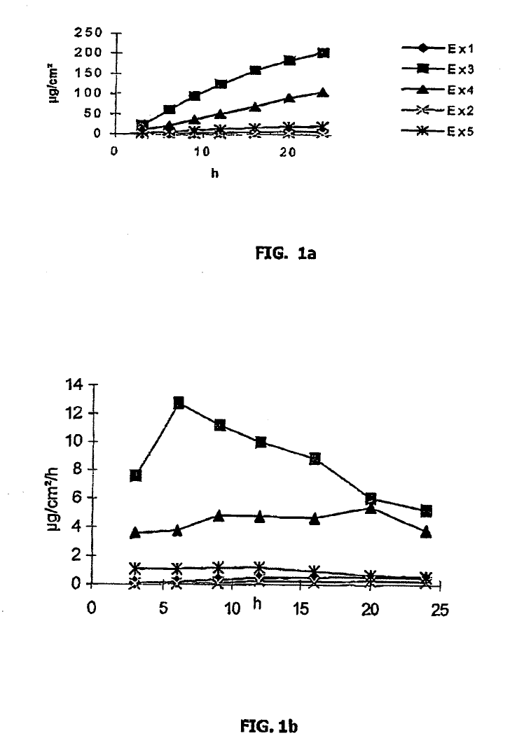

FIGS. 1a and 1b represent the quantities of dexefaroxan base released ex vivo using animal skins respectively as accumulated quantity (μg/cm²), and as flow (μg/cm²/h), in the case of transdermal formulations according to the invention.

The best flows obtained in the steady state using these formulations are between 5 and 10 μg/cm²/h. Compared with those obtained from an aqueous-alcoholic solution of the same active ingredient, they are lower, which can be explained by the very different characteristics of the galenic formulation itself. Indeed, as a general rule, the kinetics of release are more rapid using topical gel forms compared with transdermal forms.

The flow values obtained more particularly with examples 2 and 3 according to the invention confirm the possibility of envisaging a transdermal formulation based on dexefaroxan hydrochloride. Indeed, using as permeation ratio between human skin and mouse skin a commonly recognized factor of 3, and for a mean surface of the device of 25 cm², the quantity of dexefaroxan (expressed in the base form) administered to humans would be between 0.9 and 1.7 mg.

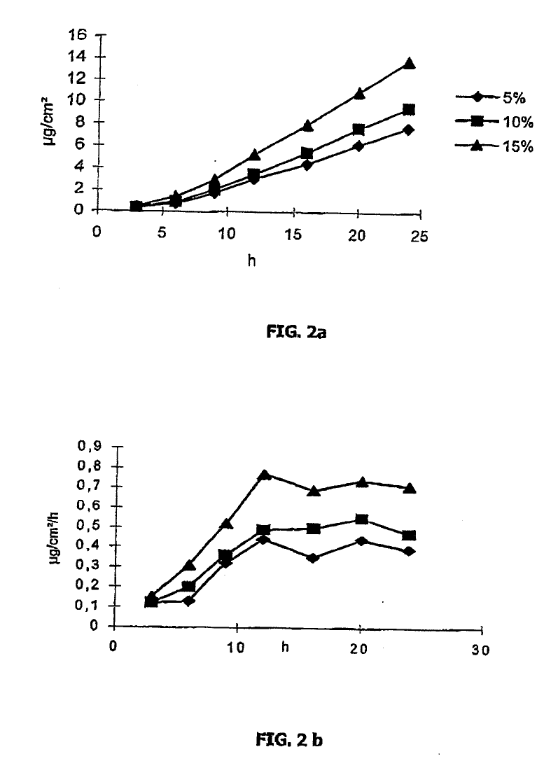

FIGS. 2a and 2b represent the quantities of dexefaroxan base released ex vivo using animal skins respectively as accumulated quantity (μg/cm²), and as flow (μg/cm²/h), in the case of transdermal formulations according to example 1 of the invention, by comparing various concentrations.

The flow values obtained more particularly with example 1 according to the invention confirm the possibility of envisaging modulating the release of the active ingredient. The quantities released at 24 hours increase significantly as a function of the dexefaroxan concentration.

To demonstrate the action of compounds capable of promoting skin permeation, permeation studies are carried out ex vivo on animal skins with the aid of a reference product whose kinetics constitutes “a blank”, and then after prior pretreatment of the skin with the promoter compound.

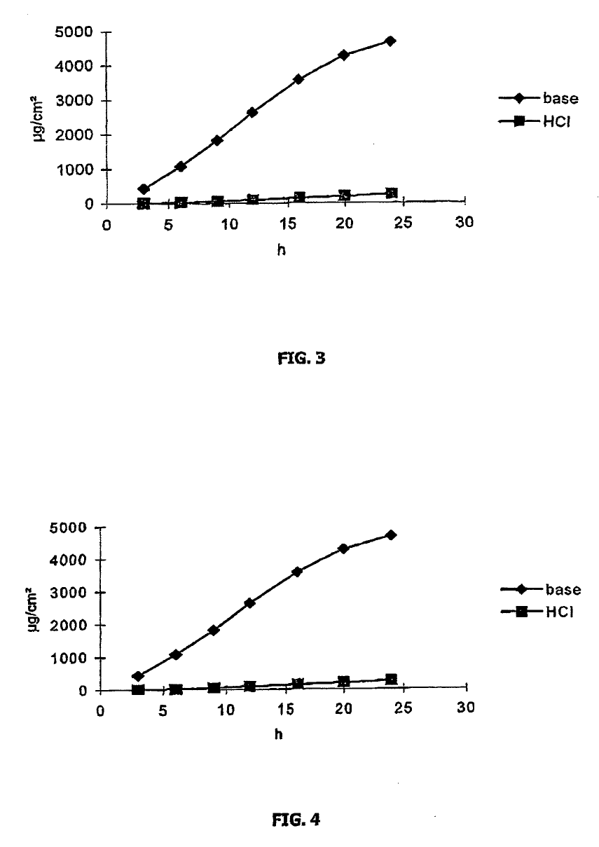

FIG. 3 represents the quantities expressed as dexefaroxan base of the accumulated quantities (μg/cm²); it involves comparing the reference batch CM586 with or without individual pretreatment with promoters.

The ratio of the quantity accumulated at 24 h of the trial to the reference makes it possible to calculate a permeation index (PI) which reflects the capacity of the test product to promote the flow expressed as dexefaroxan base; the classification of the promoters tested in increasing order of this index is the following; it demonstrates a more marked effect of polar promoters.

| | PI |
|---|---|
| N,N-Diethyl-m-toluamide | 22.2 |
| Glyceryl oleate | 9.2 |
| Oleic acid | 8.8 |
| Sorbitan monooleate | 2.6 |
| Mannitan oleate | 2.4 |

FIG. 4 represents the quantities of idazoxan base and hydrochloride released ex vivo using animal skins, in the case of aqueous alcoholic solutions according to the invention.

The quantities released at 24 h are significantly higher for the base form of idazoxan, which confirms the good skin permeation potential of this molecule.

1. A topical pharmaceutical preparation, characterized in that it consists of the combination of an oxan and a pharmaceutically acceptable excipient allowing transdermal administration of said oxan.

2. The topical pharmaceutical preparation as claimed in claim 1, characterized in that it contains, as active ingredient, an imidazoline derivative having antagonist properties for the α₂-adrenergic receptors, and in particular idazoxan, alkoxyidazoxans, a fluorinated benzodioxaneimidazoline derivative, a 2,3-dihydroxybenzofuran derivative which is disubstituted at the 2-position such as efaroxan or dexefaroxan, it being possible for said abovementioned active ingredients to be used in their racemic form or in the form of various enantiomeric mixtures, in the form of the free base or of addition salts with a pharmacologically acceptable acid such as the hydrochloride.

3. The topical pharmaceutical preparation as claimed in claims 1 and 2, characterized in that it is provided in the form of a cream, an ointment, a gel, a film-forming aerosol or spray dispenser.

4. The topical pharmaceutical preparation as claimed in claims 1 and 2, characterized in that it is provided in the form of a transdermal matrix patch.

5. The transdermal patch as claimed in claim 4, comprising a support, a protectant and a self-adhesive matrix, characterized in that said matrix comprises:
a) 40 to 95 parts by weight of at least one adhesive copolymer consisting of an active self-adhesive matrix ASAM;
b) 5 to 40 parts by weight of processing plasticizers or solvents;
c) 5 to 20 parts by weight of an oxan;
d) 0 to 20 parts by weight of solubilizing agent and/or permeation adjuvant.

6. The matrix device as claimed in claim 5, characterized in that the adhesive copolymer is composed either of one or more adhesive polyacrylates, or of one or more hydrophilic polymers, or of silicone polymers.

7. The matrix device as claimed in claim 6, characterized in that the adhesive copolymer is an adhesive acrylic copolymer consisting of at least two of the monomers chosen from the products designated hereinafter, acrylic acid, butyl acrylate, 2-ethylhexyl acrylate, vinyl acetate, methyl acrylate, glycidyl methacrylate, 2-hydroxyethyl acrylate, methyl methacrylate, n-vinylpyrrolidone, butyl methacrylate, methacrylic esters and dimethylaminoethyl methacrylate.

8. The matrix device as claimed in claim 6, characterized in that the hydrophilic polymer is obtained by combining polyvinyl alcohol and polyvinylpyrrolidone.

9. The matrix device as claimed in claim 6, characterized in that the plasticizing adjuvant forms part either of the family of mineral oils such as glycerol, or of products obtained by polymerization of ethylene, and preferably of the polyethylene glycol type having a molecular mass of between 200 and 8 000.

10. The matrix device as claimed in claim 6, characterized in that the polymer is of the polydimethylsiloxane type, obtained by condensation of a silanol with a silicate resin.

11. The matrix device as claimed in any one of claims 1 to 10, characterized in that its matrix comprises, for a total of 100 parts by weight:
a) 60 to 80 parts by weight of an adhesive and self-crosslinkable acrylic copolymer, in the form of a solution containing about 47.5% w/v of 2-ethylhexyl acrylate, glycidyl methacrylate, 2-hydroxyethyl acrylate and vinyl acetate copolymer and, as crosslinking agent, polybutyl titanate, said “ready-to-use” adhesive copolymer having a glass transition temperature of −50° C.;
b) 0.5 to 10 parts by weight of polyvidone;
c) 5 to 15 parts by weight of dexefaroxan in hydrochloride form;
d) 0 to 2 parts by weight of antioxidant;
e) 0 to 15 parts by weight of one or more absorption promoters.

12. The matrix device as claimed in any one of claims 1 to 10, characterized in that its matrix comprises, for a total of 100 parts by weight:
a) 60 to 80 parts by weight of an adhesive and self-crosslinkable acrylic copolymer, in the form of a solution containing about 47.5% w/v of acrylic acid, butyl acrylate, 2-ethylhexyl acrylate and vinyl acetate copolymer and, as crosslinking agent, aluminum acetylacetonate, said “ready-to-use” adhesive copolymer having a glass transition temperature of −50° C.;
b) 0.5 to 10 parts by weight of polyvidone;
c) 5 to 15 parts by weight of dexefaroxan in hydrochloride form;
d) 0 to 2 parts by weight of antioxidant;
e) 0 to 15 parts by weight of one or more absorption promoters.

13. The matrix device as claimed in any one of claims 1 to 10, characterized in that its matrix comprises, for a total of 100 parts by weight:
a) 30 to 60 parts by weight of an adhesive acrylic copolymer, in the form of a solution containing about 60% w/v of dimethylaminoethyl methacrylate and methacrylic ester copolymer, and, as crosslinking agent, succinic acid, and, as plasticizer, acetyltributyl citrate;
b) 0.5 to 10 parts by weight of polyvidone;
c) 5 to 10 parts by weight of dexefaroxan in hydrochloride form;
d) 0 to 2 parts by weight of antioxidant;
e) 0 to 15 parts by weight of one or more absorption promoters.

14. The matrix device as claimed in any one of claims 1 to 10, characterized in that its matrix comprises, for a total of 100 parts by weight:
a) 5 to 20 parts by weight of polyvinyl alcohol;
b) 20 to 60 parts by weight of polyvidone;
c) 10 to 30 parts by weight of glycerol;
d) 10 to 30 parts by weight of polyethylene glycol;
e) 5 to 15 parts by weight of dexefaroxan in hydrochloride form;
f) 0 to 2 parts by weight of antioxidant;
g) 0 to 15 parts by weight of one or more absorption promoters.

15. The matrix device as claimed in any one of claims 1 to 10, characterized in that its matrix comprises, for a total of 100 parts by weight:
a) 70 to 95 parts by weight of silicone polymer;
b) 5 to 15 parts by weight of dexefaroxan in hydrochloride form;
c) 0 to 2 parts by weight of antioxidant;
d) 0 to 15 parts by weight of one or more absorption promoters.

16. The matrix device as claimed in claim 1, characterized in that the topical formulation comprises, for a total of 100 parts by weight:
a) 5 to 30 parts by weight of polysaccharide polymer;
b) 1 to 15 parts by weight of dexefaroxan in hydrochloride form;
c) 5 to 30 parts by weight of ethanol;
d) 20 to 60 parts by weight of water;
e) 0 to 15 parts by weight of one or more absorption promoters.

17. The matrix device as claimed in claim 1, characterized in that the topical formulation comprises, for a total of 100 parts by weight:
a) 2 to 40 parts by weight of carbomer;
b) 1 to 15 parts by weight of dexefaroxan in hydrochloride form;
c) 5 to 25 parts by weight of isopropyl alcohol;
d) 5 to 25 parts by weight of polyoxyethylene alkyl ether;
e) 5 to 25 parts by weight of fatty acid esters;
f) 5 to 25 parts by weight of fatty alcohol esters;
g) 0 to 15 parts by weight of one or more absorption promoters.

18. The matrix device as claimed in any one of claims 1 to 17, characterized by the presence of one or more absorption promoters, preferably selected from alcohols, glycols, polyglycols, amides of the pyrrolidone type and derivatives, surfactants of the nonionic type, polysorbates, alkyl ethers, aryl ethers, poloxamers, saturated or unsaturated fatty acids with a carbon chain between C₅ and C₃₀, fatty alcohols, polyglycosylated glycerides, alone or as mixtures, glycol esters of propylene glycol or of polyglycerol, fatty acid esters of the polyol type, alkylglyceryl ether, propylene glycol, glycerine, polyoxyethylene glycerol, polyglycerol, sorbitan, polyoxyethylene sorbitan, polyoxyethylene castor oil, alkyl ether, esters of sugars, derivatives of collagens, terpenic essential oils, compounds of the m-diethyltoluamide type, antipuriginous compounds of the crotamiton type, compounds of the phospholipid type, lecithin derivatives, neohesperidin dihydrochalcone derivatives.

19. A method for preparing an adhesive matrix device as claimed in any one of claims 1 to 12, characterized in that it comprises the following steps:
preparing a premixture of active ingredient in the cosolvent(s) for the adhesive or in an additional processing solvent, in order to obtain either a solution, or a dispersion;
adding to the preceding premixture the required quantities of plasticizers and adhesive;
placing the mixture directly on a supporting film, preferably of the silicone polyester type, so as to obtain a layer having a thickness of between 50 and 100 g/m² (expressed as dry weight);
drying the coating thus obtained in order to evaporate the processing solvents and to allow crosslinking of the polymers, by progressive drying at a temperature of between 50° C. and 110° C., and preferably via different drying methods;
pasting onto the dried coating an occlusive film, for example of the polyester type;
cutting out to the desired surface and packaging in a sachet.

20. The use of an oxan as defined in claim 2 for the manufacture of a topical pharmaceutical preparation intended for the treatment of lipolysis and of obesity.

21. The use of an oxan as defined in claim 2, for the manufacture of a transdermal matrix patch intended for the treatment of Alzheimer's disease, of progressive supranuclear paralysis (PSP), of Parkinson's disease and of depression.
